(19) United States
(12) Patent Application Publication
(10) Pub. No.: US 2016/0029923 A1
(43) Pub. Date: Feb. 4, 2016

(54) NASOPHARYNGEAL OR OROPHARYNGEAL CANNULA FOR MAIN-STREAM CAPNOGRAPHY

(71) Applicant: DELTAMEDICS, Gouvieux (FR)

(72) Inventor: Jean-Christophe Ozenne, Gouvieux (FR)

(73) Assignee: Deltamedics, Gouvieux (FR)

(21) Appl. No.: 14/776,428

(22) PCT Filed: Mar. 13, 2014
(86) PCT No.: PCT/EP2014/054941

§ 371 Date: Sep. 14, 2015

(30) Foreign Application Priority Data
Mar. 15, 2013 (FR) 1352332

(51) Int. Cl. A61B 5/097; A61B 5/00; A61M 16/08; A61M 16/06; A61B 5/083

(52) U.S. Cl. CPC A61B 5/097; A61M 16/0666; A61B 5/0836; A61M 16/085; A61M 16/0683; A61B 5/0086; A61M 2202/0208; A61M 2202/0014

(57) ABSTRACT

The invention relates to a nasopharyngeal or oropharyngeal cannula (1, 10) comprising a body (2, 20) formed by a tube (4, 40) extending through a collar (3, 30) which can rest on the outer edges of a buccal or nasal cavity of a patient, said tube comprising a first portion (43, 430) that is at least partially curved so as to be inserted into the patient's buccal or nasal cavity as far as the pharynx and a main channel (5, 50) forming a fluid passage between the two ends (41, 42; 410, 420) of the tube. The tube comprises a hard second portion (44, 440) disposed on one side of the collar opposite the first portion, said second portion comprising a notch (E) transverse to the axis of the main channel and configured to receive infrared means that can obtain a capnography measurement of the air exhaled through the main channel.

The invention relates to a device for clearing the airways of a patient, more particularly an oropharyngeal or nasopharyngeal cannula allowing mainstream capnographic measurement.

A cannula is a straight or curved, flexible or rigid tube which permits the passage of a fluid such as air or a liquid through an orifice.

An oropharyngeal or nasopharyngeal cannula is used in medicine to keep open the airways of an unconscious or semiconscious patient, for example a patient who is in a coma, under anesthesia or in the process of waking. More specifically, an oropharyngeal or nasopharyngeal cannula makes it possible to maintain the permeability of the airways with the hypopharynx and to facilitate the removal of tracheobronchial secretions.

A nasopharyngeal cannula is generally in the form of a flexible tube made of plastic and in two parts.

A first part is curved slightly and flexible, or semi-rigid, so as to follow the trajectory of the nasal cavity as far as the pharynx and to thus clear a channel as far as the pharynx. The free end of the first part is positioned behind the base of the tongue, above the epiglottis. In this way, a passage of air is maintained toward the lungs.

A second part comprises a collar, which rests on the entrance to the patient's nostril once the cannula is in place and which avoids any excessive forward movement of the cannula.

An oropharyngeal cannula, for its part, is generally in the form of a Guedel cannula or Mayo cannula. The Guedel cannula is formed by a semi-rigid tube of plastic and is in three parts.

A first part is curved so as to follow the trajectory of the oral cavity as far as the pharynx. Its shape makes it possible in particular to push forward the lingual mass, moving the latter away from the posterior wall of the pharynx, thereby freeing a channel between the tongue and the palate of the patient as far as his pharynx. In this way, a passage of air is maintained toward the lungs, and the tongue is held in position so that it does not sink rearward onto the epiglottis and obstruct the airways, in particular when the unconscious patient is lying on his back.

A second, straight part is reinforced in the interior with a rigid ring which is designed to be positioned in the dental area. The rigidity of this area makes it possible to keep the orifice open when the cannula is placed between the teeth, preventing an intubated patient from obstructing an orotracheal tube by biting, for example.

A third part comprises a collar which rests on the patient's lips once the cannula is in place.

There are several sizes of oropharyngeal or nasopharyngeal cannula. The sizes vary between children and adults. Their size is expressed mainly as a function of the internal diameter of the cannula. For oropharyngeal cannulas, the sizes also vary according to the distance between the corner of the lips and the angle of the jaw in children and adults. In adults, it is also possible to use the distance between the corner of the lips and the earlobe.

During an operation under anesthesia, a face mask is generally used in order to inject dioxygen into the patient via the conduit of the oropharyngeal cannula or nasopharyngeal cannula. However, the use of a mask impedes access to the patient's face, if this is necessary.

In addition, the use of a mask gives rise to complex positioning in order to be able to carry out capnography of the air exhaled by the patient.

Capnography is a measurement of the concentration or partial pressure of carbon dioxide in the air exhaled by a patient. Measurements of this type are commonly used on patients under anesthesia. The presence of carbon dioxide in the air exhaled over several exhalations by a patient who has just been intubated makes it possible in particular to confirm that the endotracheal tube is properly placed in the trachea.

Capnography also makes it possible to obtain an indirect measurement of the partial pressure of carbon dioxide in the arterial blood. This information makes it possible to evaluate the state of vascularization of the patient. Capnography reflects directly the capacity of the patient's lungs to eliminate carbon dioxide, and it reflects indirectly the production of carbon dioxide by the tissues and its transport as far as the lungs.

It makes it possible to detect, very early on, signs of respiratory deficiency such as hypoventilation, or disconnection of a circuit or tube in the esophagus. During an operation under anesthesia, capnography makes it possible to provide information such as the frequency and regularity of ventilation, which is more useful than the information provided by an oximeter.

It provides a method for rapid detection of critical conditions, such as a badly positioned tracheal tube, a ventilation defect, or a circulatory defect, and for prevention of irreversible complications.

In order to carry out both injection of dioxygen and collection of the gases exhaled by the patient, so as to perform a measurement of the level of carbon dioxide, without using a cumbersome face mask, the document US 2008/0000481 discloses an oropharyngeal device with a shape which is designed to be inserted in a patient's mouth. The device comprises a body having at least two channels which extend through the body in order to form two air passages through which, firstly, dioxygen can be injected, and, secondly, carbon dioxide can be removed. The channels are formed in the body of the device, which comprises a collar on the proximal portion in order to prevent the device from being introduced too far into the mouth.

These uniquely oropharyngeal devices do not have means for easily and quickly securing them on the patient, which means would allow them to be maintained on the patient once the device has been fitted in the patient's mouth, while permitting rapid removal of the device if the patient wakes up or if intubation is necessary.

There is also known from documents US 2007/267024, US 2008/308108, U.S. Pat. No. 4,821,715 and EP 1 188 457 an oropharyngeal or nasopharyngeal device which is designed to form a passage for gas to be inhaled, in which there is accommodated a pair of conduits which are designed to be slid into the inner passage(s) in order to inject dioxygen, in particular over a proximal portion, and to collect exhaled gas, in particular over a distal portion, in order to measure the level of CO₂.

These devices also do not have means for easily and quickly securing them on the patient, which means would allow them to be maintained on the patient once the device has been fitted in the patient's mouth or nose, whilst permitting rapid removal of the device if the patient wakes up or if intubation is necessary.

Moreover, these devices have the disadvantage of being in several parts, namely a part composed of the cannula, and a part composed of the injection and extraction tubes to be inserted. The additional assembly time may be critical, in particular in an emergency situation.

An oropharyngeal or nasopharyngeal cannula has to be fitted and secured on the patient in such a way that he is able to remove it when necessary, especially when the patient wakes up. The securing of the cannula makes it possible to hold the device in position, but this securing must be easily removable.

An oropharyngeal cannula such as a Guedel cannula can cause vomiting in a conscious patient. A patient regaining consciousness will spontaneously cough out the cannula when he recovers the cough reflex. The cannula can also be replaced at a given moment by an intubation device.

A nasopharyngeal cannula is most often put in place during the period of recovery after anesthesia, in order to facilitate clearance of the bronchi, and also in cases where the patient is semi-conscious. Although a nasopharyngeal cannula is generally better tolerated by a patient than an oropharyngeal cannula of the Guedel type, some patients may feel a degree of discomfort that may lead to the nasopharyngeal cannula being removed urgently.

In addition, in all the prior art documents cited above, capnography is carried out using a side stream, not the main stream. The main stream corresponds to the flow of exhaled fluid through the main conduit of the oropharyngeal cannula or nasopharyngeal cannula. A side stream corresponds to a flow withdrawn via an auxiliary conduit that has a cross section smaller than the cross section of the main conduit. The flow rate of a side stream is consequently less than that of the main stream.

Moreover, the document US 2007/0095347 discloses a naso-pharyngeal device designed both to inject dioxygen and also to collect the gases exhaled by the patient, in order to measure the level of carbon dioxide without using a cumbersome face mask.

However, such a device can only be used nasally and does not use a nasopharyngeal cannula, so that the airways may be obstructed, which means it is not possible to guarantee optimal injection of oxygen and collection of carbon dioxide under the best possible conditions.

Furthermore, the device used is heavy and very bulky in the area of the patient's nose, and it has a complex harness for fitting on the patient.

A nasal cannula for injecting dioxygen into the patient's nostrils is also known from the document U.S. Pat. No. 3,802,431. The device is composed of a small cannula placed at the entrance to each nostril in order to inject dioxygen into each nostril with the aid of tubes that are connected to the cannulas and are sufficiently flexible to be passed behind the patient's ears once the device is in place on the patient.

However, such a device does not have a nasopharyngeal cannula and only permits the injection of a fluid at the entrance to the nostrils.

The object of the invention is to eliminate the aforementioned disadvantages by making available an oropharyngeal or naso-pharyngeal cannula which is designed to be secured on the patient, in particular with the aid of the tubes for injection/extraction, and to keep the cannula in place on the patient and perform main-stream capnography.

According to one aspect of the invention, in one embodiment, an oropharyngeal or nasopharyngeal cannula is proposed comprising a body formed by a tube extending through a collar that is able to rest on the outer edges of an oral or nasal cavity, respectively, of a patient, the tube comprising, on one side of the collar, a first portion that is at least partially curved so as to be inserted into the oral or nasal cavity, respectively, of the patient as far as the pharynx, and a main conduit forming a fluid passage between the two ends.

According to a general feature of the invention, the tube comprises a rigid second portion arranged on the side of the collar opposite the first portion, the second portion having a notch which is transverse with respect to the axis of the main conduit and which is configured to receive infrared means that can perform a capnographic measurement of the air exhaled through the main conduit.

The second portion of the tube, protruding outward from the collar when the cannula is fitted on the patient, makes it possible to mount, in the notch provided, a known infrared device for measuring the level of carbon dioxide present in the air exhaled by the patient through the main conduit.

Advantageously, the collar can comprise two input/output orifices which are oriented in a radial direction with respect to the axis of the main conduit in such a way as to be oriented laterally on each side of the oral or nasal cavity of the patient when the cannula is fitted on the patient, the two input/output orifices being connected to one or two auxiliary conduits which are formed in the body and extend from the collar to the main conduit via the first portion of the tube.

The thickness of the collar must have a thickness sufficient to permit the passage of the auxiliary conduits.

The two input/output orifices connected to the same auxiliary conduit, or each connected to its own auxiliary conduit, thus make it possible to inject dioxygen into the patient's airways via the oropharyngeal or nasopharyngeal cannula without using any means other than the cannula, in particular without using a mask.

Moreover, by thus orienting the input/output orifices radially on the collar so as to be on either side of the oral or nasal cavity of the patient when the cannula is fitted, the injection/extraction tubes, which are coupled to the input/output orifices, extend in the direction of the patient's ears, on each side of the patient's nostrils. In this way, each injection/extraction tube can be passed behind an ear in order to secure the cannula on the patient such that it is easily removable.

Preferably, the two input/output orifices arranged radially on the collar are connected to an auxiliary conduit via a portion of the auxiliary conduit extending radially in the collar, each forming an angle of between 0° and 20°, more particularly an angle of about 10°, with the plane defined by the surface of the collar, so as to orient the input/output orifices toward the patient's face.

This angle makes it possible to orient the injection/extraction tubes so as to keep them as close as possible to the patient's face, and thus to reduce the risks of an element or a tool getting caught in the injection/extraction tubes.

The cannula can advantageously comprise two flexible injection/extraction tubes which can be passed behind the patient's ears when the cannula is fitted on the patient, a first end of each tube being connected to an input/output orifice.

The injection/extraction tubes can be connected to the input/output orifices in a removable manner, or securely and non-removably. The presence of the integral tubes on the oropharyngeal cannula makes it possible to avoid any assembly operation apart from the connection of the free ends of the tubes to extraction means, such as a capnograph, or to injection means during the fitting of the cannula on the patient.

The cannula can also comprise a clamping ring which is able to hold the two injection/extraction tubes together and to slide along the two injection/extraction tubes so as to hold the cannula in place when it is fitted on the patient and each injection/extraction tube passes behind an ear.

Once the injection/extraction tubes have been passed behind each ear, the clamping ring makes it possible to clamp the tubes under the chin by lifting the clamping ring toward the chin. This makes it possible to securely fasten the cannula, while at the same time making it possible to quickly remove the cannula if necessary.

Preferably, a second end of the injection/extraction tubes comprises means for connection to injection or extraction appliances.

The connection means can be specific connectors which are dedicated to appliances for measurement or injection of gas. By having a specific connector for each tube, the risks of connection error are zero, even in an emergency situation, since each connector is dedicated to a specific appliance.

In one embodiment, the cannula comprises a single auxiliary conduit which extends as far as a proximal part of the first portion of the tube and is connected via the two input/output orifices to the injection/extraction tubes, said injection/extraction tubes being tubes for injection of dioxygen.

The injection of dioxygen with the aid of the cannula makes it possible to avoid the use of a mask, which would prevent or at least impede access to the patient's face.

According to one variant, the cannula comprises a first auxiliary conduit which extends as far as a proximal part of the first portion of the tube, and a second conduit extending as far as a distal part of the first portion of the tube, the first auxiliary conduit being connected via an input orifice to an injection tube for dioxygen, and the second auxiliary conduit being connected to an output orifice, which is connected to a tube for aspiration of the secretions in the airways of the patient.

According to this variant, the second auxiliary conduit can be formed in an upper wall of the first portion of the tube.

By forming the second auxiliary conduit in the upper wall of the tubular portion, the ability to aspirate secretions such as mucus and saliva is optimized. Indeed, when the patient is lying on his back, the upper surface of the distal wall of the oropharyngeal or nasopharyngeal cannula, fitted in the patient's nose as far as his pharynx, is located in the area of the free end near the back of the throat where the secretions can accumulate.

Advantageously, the auxiliary conduit, or at least one of the auxiliary conduits when there are two of them, can comprise a cross section of oblong shape, at least on part of the first portion of the tube.

In this way, the volume of gaseous or liquid fluid that can be conveyed through the conduit is greater than in the case of a conduit with a cylindrical cross section.

Moreover, with a body comprising a main conduit with an oblong cross section, that is to say similar to the shape of a mouth, as is the case for oropharyngeal cannulas, the auxiliary conduits can be formed in the upper wall and the lower wall, without having to substantially increase the thickness of the walls of the body of the cannula. This can be done while maintaining a substantial volume of transferred gas.

In the case of nasopharyngeal cannulas, in which the tube generally has a cylindrical cross section, the oblong cross sections can be formed in such a way as to follow the contours of the main conduit of the nasopharyngeal cannula. In this way, curved oblong cross sections are obtained, which makes it possible to form the auxiliary conduits in the walls of the cannula without having to substantially increase the thickness of the walls of the body of the cannula. This can be done while maintaining a substantial volume of transferred gas.

Preferably, at least one of the auxiliary conduits comprises an orifice which opens into the main conduit and has a frustoconical shape, the orifice having a larger cross section than the mean cross section of the auxiliary conduit.

The frustoconical shape of the orifice that opens into the main conduit at the level of the tubular portion of the body makes it possible to assist the good distribution of the dioxygen injected in one case, and to optimize the aspiration of the secretions in another case.

In one embodiment of the invention for a nasopharyngeal cannula, the direction defined by an input/output orifice and the axis of the main conduit forms an angle of between −10° and 20°, more particularly between 0° and 10°, with an axis orthogonal to the axis of the patient's nose when the cannula is fitted on the patient.

An angle of between −10° and 20°, particularly an angle of 0°, makes it possible to direct the injection/extraction tubes of the nasopharyngeal cannula toward the upper edge of the ears, thus making it easier to pass the tubes behind the ears.

Advantageously, the free end of the first portion of the tube of the nasopharyngeal cannula can be provided with a rounded shape. The rounded shape of the free end makes it possible to reduce the risks of injury when inserting the nasopharyngeal cannula, especially compared to a nasopharyngeal cannula that has a beveled free end.

The body can additionally be made of a flexible and sliding plastic. In this way, the nasopharyngeal cannula can be inserted without using a lubricating gel.

In one embodiment of the invention for an oropharyngeal cannula, the direction defined by an input/output orifice and the axis of the main conduit forms an angle of between 0° and 30°, more particularly between 10° and 20°, with an axis passing through the corners of the patient's lips when the cannula is fitted on the patient.

An angle of between 10° and 20°, particularly an angle of 15°, makes it possible to direct the injection/extraction tubes toward the upper edge of the ears, thus making it easier to pass the tubes behind the ears.

Advantageously, the oropharyngeal cannula can comprise an additional conduit formed in the collar and extending from a portion of an auxiliary conduit as far as two additional output orifices that are formed in an upper part of the collar, the two additional output orifices being arranged so as to be opposite each of the patient's nostrils when the cannula is fitted on the patient.

Other advantages and features of the invention will become clear from examination of the detailed description of various non-limiting embodiments and from the attached drawings, in which:

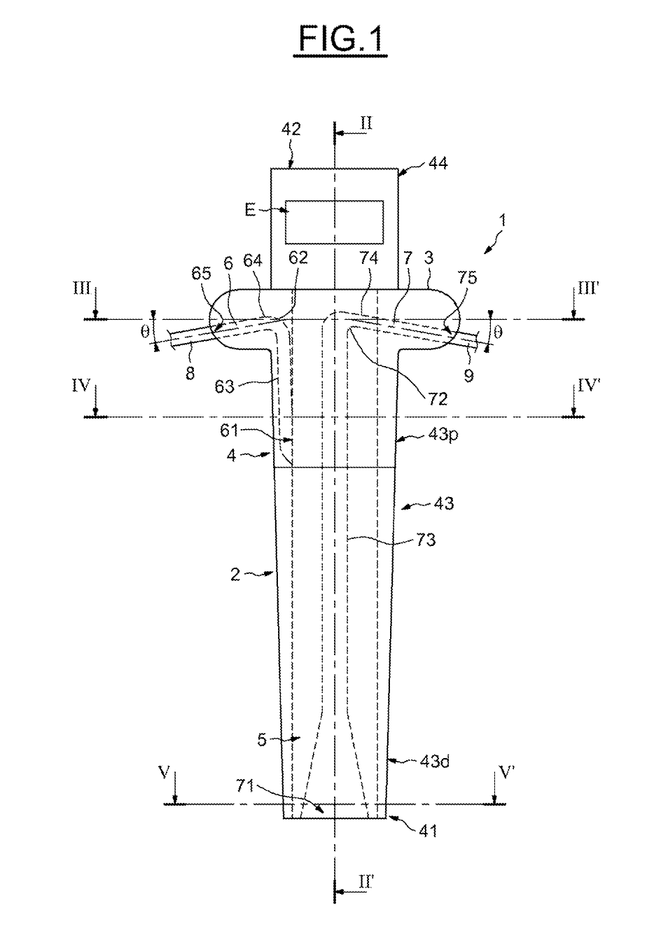

FIG. 1 shows a schematic plan view of an oropharyngeal cannula according to an embodiment of the invention;

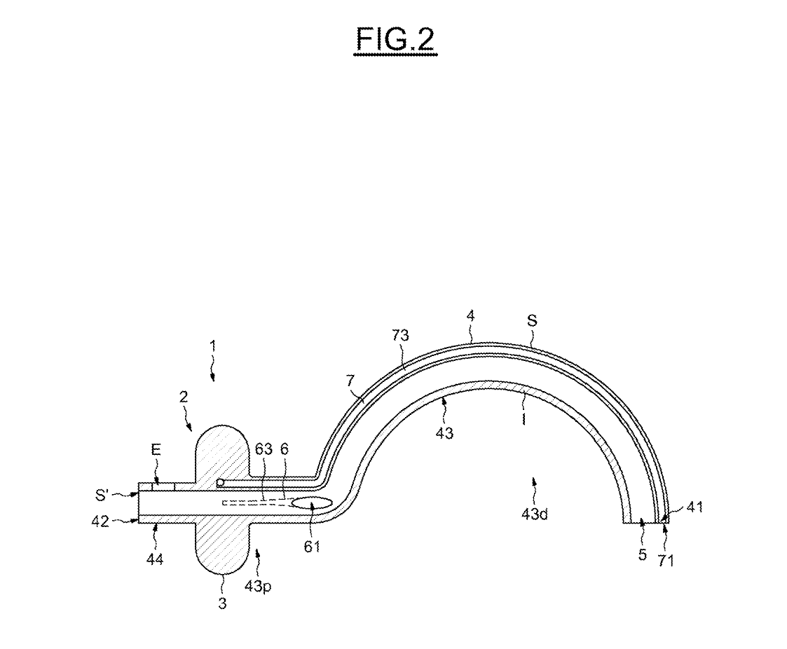

FIG. 2 shows a sectional view of the oropharyngeal cannula from FIG. 1 in a longitudinal plane II-II′;

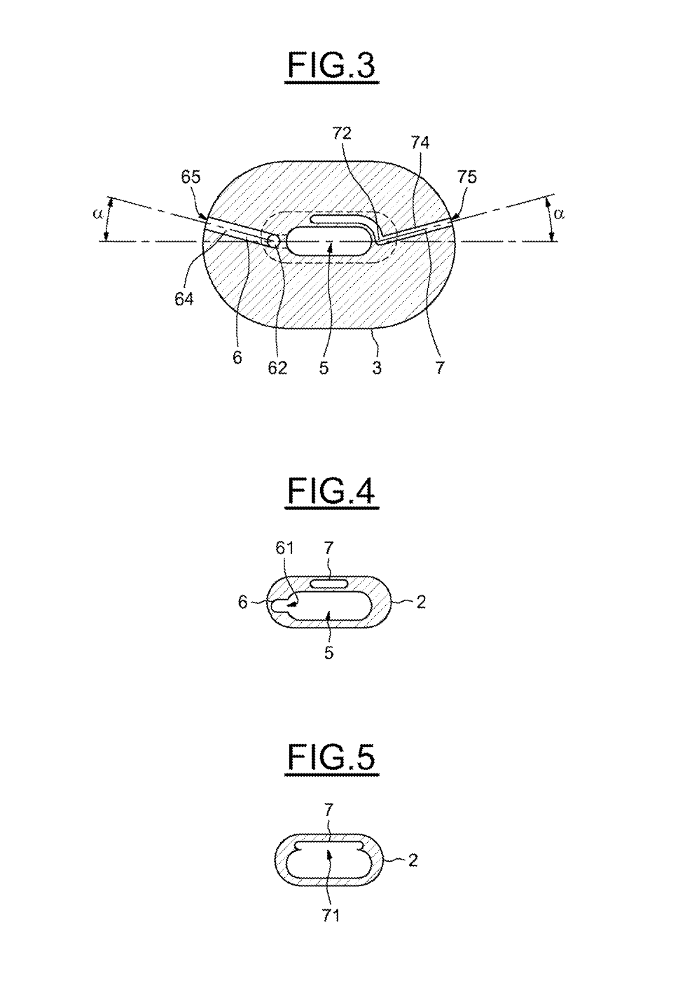

FIG. 3 shows a sectional view of the oropharyngeal cannula from FIG. 1 in a transverse plane III-III′;

FIG. 4 shows a sectional view of the oropharyngeal cannula from FIG. 1 in a transverse plane IV-IV′;

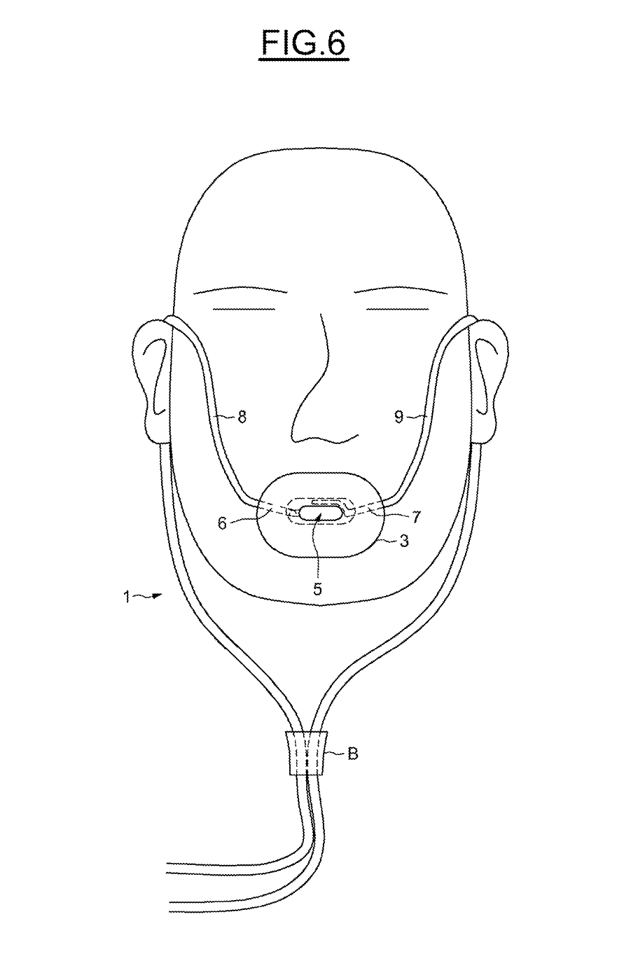

FIG. 5 shows a sectional view of the oropharyngeal cannula from FIG. 1 in a transverse plane V-V′;

FIG. 6 shows a schematic representation of an oropharyngeal cannula from FIG. 1 fitted on a patient;

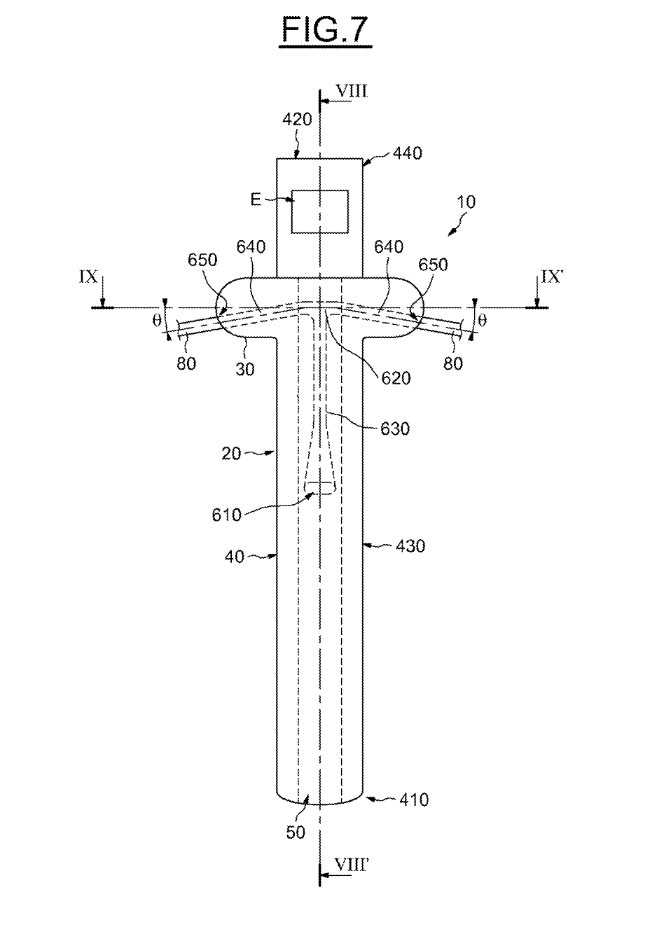

FIG. 7 shows a schematic plan view of a nasopharyngeal cannula according to an embodiment of the invention;

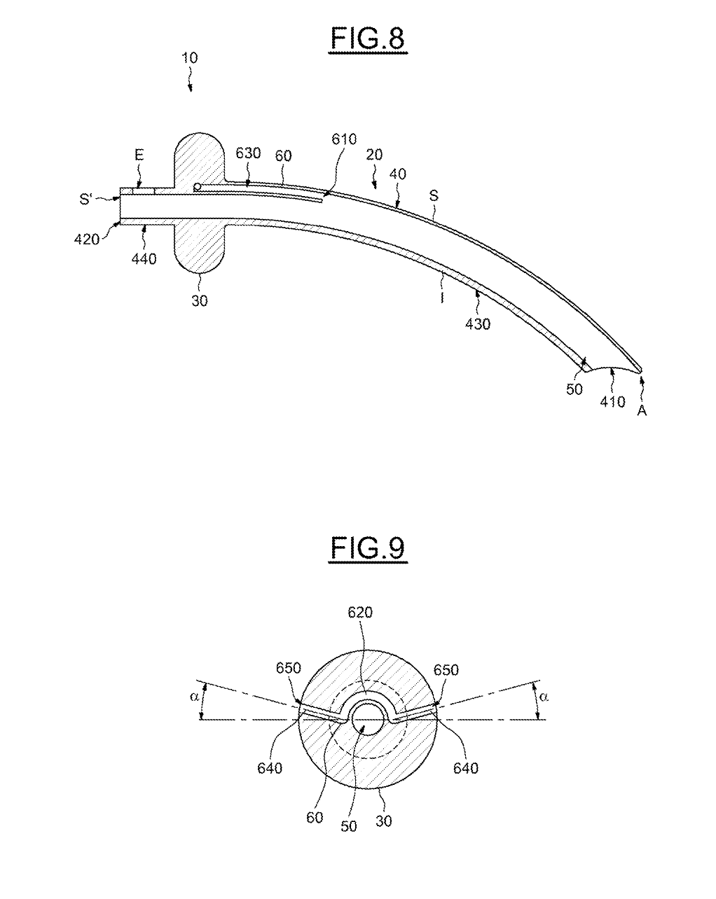

FIG. 8 shows a sectional view of the nasopharyngeal cannula from FIG. 7 in a longitudinal plane VIII-VIII′;

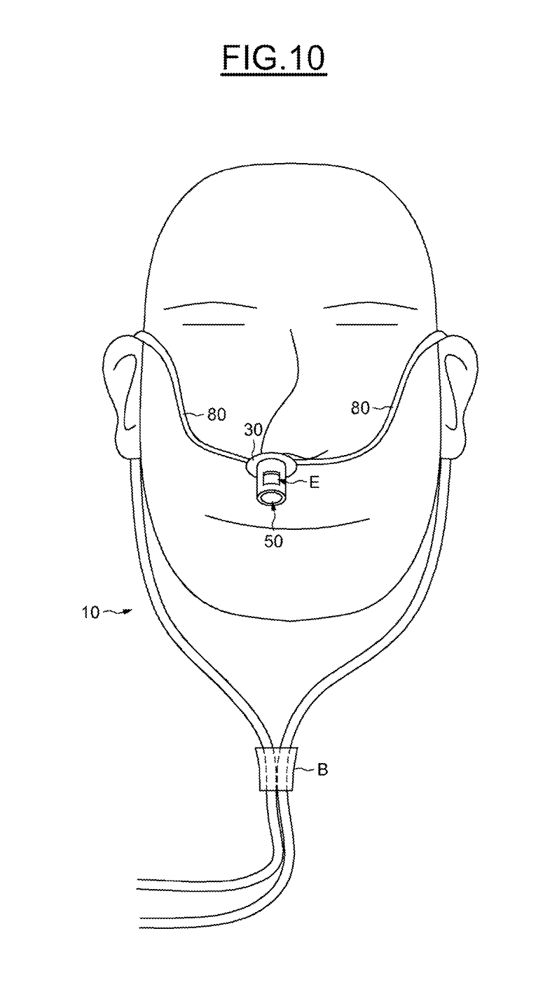

FIG. 9 shows a sectional view of the nasopharyngeal cannula from FIG. 7 in a transverse plane IX-IX′;

FIG. 10 shows a schematic representation of a nasopharyngeal cannula from FIG. 7 fitted on a patient.

FIGS. 1 to 6 are schematic illustrations of an oropharyngeal cannula 1 according to an embodiment of the invention.

FIG. 1 shows a schematic plan view of the oropharyngeal cannula 1.

The oropharyngeal cannula 1 comprises a body 2 made of rigid plastic and formed with a collar 3 through which a tube 4 passes. The tube 4 comprises two free ends 41 and 42 between which extend a first portion 43 and a second portion 44 which are arranged on either side of the collar 3.

As is illustrated in FIG. 2, which shows the oropharyngeal cannula 1 from FIG. 1 in longitudinal section in the plane II-II′, the first portion 43 of the tube 4 comprises a straight proximal part 43p, and a distal part 43d of curved shape.

The distal part 43d is curved so as to allow the oropharyngeal cannula 1, and in particular its first portion 43, to be inserted into a patient's mouth and so as to immobilize the lingual mass at the front, and thereby keep the pharynx open.

The body 2 comprises a main conduit 5 extending between the two free ends 41 and 42 of the tube 4. The main conduit 5 thus forms a fluid passage between the outside and the airways of the patient, and more particularly his pharynx, when the oropharyngeal cannula 1 is fitted.

The collar 3 is intended to rest on the patient's lips once the oropharyngeal cannula 1 is inserted into the patient's mouth, while the proximal part 43p situated between the collar 3 and the distal part 43d is located in the area of the patient's teeth. The proximal part 43p can therefore be reinforced, for example by making this part from a more rigid material than the rest of the body 2, in order to avoid any risk of biting, which could give rise to obstruction of the main conduit 5. In order to reinforce the proximal part 43p, a sleeve (not shown) can also be inserted into the main conduit 5 in such a way as to extend from the collar 3 to the proximal part 43p, the sleeve being hollow on its central axis in order to maintain a fluid passage in the main conduit 5.

As is illustrated in FIGS. 1 and 2, the oropharyngeal cannula 1 in this embodiment comprises a first auxiliary conduit 6 and a second auxiliary conduit 7, which are formed within the thickness of the body 2 separating the main conduit 5 from the exterior of the body 2.

The first auxiliary conduit 6 extends from the collar 3 into the proximal part 43p in order to open into the main conduit 5 via a first opening 61, as is shown in FIGS. 1 and 2, and also in FIG. 4 which shows a view of the oropharyngeal cannula 1 from FIG. 1 in the sectional plane IV-IV′.

As is shown in FIG. 1 and also in FIG. 3, which shows the oropharyngeal cannula 1 from FIG. 1 in a sectional view in the plane III-III′, the first auxiliary conduit 6 comprises a bend 62 which connects a first portion 63 of the first auxiliary conduit 6, extending in the first portion 43 of the tube 4 parallel to the transverse axis II-II′ as far as the first opening 61, and a second portion 64 of the first auxiliary conduit 6 extending in the collar 3.

The second portion 64 extends in the collar 3 as far as an input orifice 65, thus forming an angle of about 10° with the plane defined by the surface of the collar 3. This orientation of the second portion 64 makes it possible to orient a dioxygen injection tube 8, connected to the input orifice 65, toward the patient's face and thus optimize the maintenance in position of the oropharyngeal cannula 1.

In order to optimize the injection of dioxygen into the main conduit 5, the first auxiliary conduit 6 has a frustoconical shape at its end opening into the first opening 61. The cross section of the first opening 61 is therefore larger than the cross section of the first auxiliary conduit 6. This frustoconical shape makes it possible to assist the distribution of the fluid injected into the main conduit 5.

The second auxiliary conduit 7 extends from the collar 3 to the free end 41 of the first portion 43 of the tube 4 in order to open into the main conduit 5 via a second opening 71, as is shown in FIGS. 1 and 2, and also in FIG. 5 which shows the oropharyngeal cannula from FIG. 1 in the sectional plane V-V′.

As is shown in FIGS. 1 to 3, the second auxiliary conduit 7 comprises a bend 72 which connects a first portion 73 of the second auxiliary conduit 7, extending in the first portion 43 of the tube 4 parallel to the transverse axis II-II′ as far as the second opening 71, and a second portion 74 of the second auxiliary conduit 7 extending in the collar 3.

Like the second portion 64 of the first auxiliary conduit 6, the second portion 74 of the second auxiliary conduit 7 extends in the collar 3 as far as an output orifice 75, thus forming an angle of about 10° with the plane defined by the surface of the collar 3, which makes it possible to orient an aspiration tube 9, connected to the output orifice 75, toward the patient's face and thus optimize the maintenance in position of the oropharyngeal cannula 1.

The second auxiliary conduit 7, connected to the aspiration tube 9, is intended to aspirate the tracheobronchial secretions from the patient when the oropharyngeal cannula 1 is fitted on the patient.

In order to optimize the aspiration, the second opening 71 is arranged at the free end 41 of the first portion 43 of the tube 4 and has a cross section larger than the cross section of the rest of the second auxiliary conduit 7. The second auxiliary conduit 7 therefore has a frustoconical shape in the area of the free end 41 of the first portion 43 of the tube 4.

In order to further improve the aspiration of the secretions via the second auxiliary conduit 7, the second auxiliary conduit 7 is formed in an upper wall S of the first portion 43 of the tube 4. In fact, when the patient is lying on his back in the area of the free end 41, the upper wall S of the first portion 43 of the oropharyngeal cannula 1 fitted in the patient's mouth is located at the back of the throat.

The connection of the injection tube 8 and of the aspiration tube 9 to the input orifice 65 and to the output orifice 75, respectively, is produced by welding or by overmolding the collar 3 onto the injection and aspiration tubes 8 and 9, or with the aid of connectors.

The free end of the injection tube 8 can comprise a specific connector, for example a standard conical connector for dioxygen, which is designed to be coupled to a device delivering a flow of dioxygen, optionally via a tube cross section reducer.

As is shown in FIG. 3, the second portion 64 of the first auxiliary conduit 6 and the second portion 74 of the second auxiliary conduit 7 each form an angle a with an axis passing through the first bend 62 and the second bend 72. The angle a has a value of about 15°. This angle allows the injection tube 8 and the aspiration tube 9 each to be oriented in the direction of the upper edge of an ear of the patient, so as to facilitate the passage of the injection and aspiration tubes 8 and 9 behind the ears, as is illustrated in FIG. 6, which shows a schematic representation of the oropharyngeal cannula 1 from FIG. 1 fitted on a patient.

The passage of the injection and extraction tubes 8 and 9 behind the patient's ears makes it possible to hold the oropharyngeal cannula 1 in place on the patient's lips. The oropharyngeal cannula 1 additionally comprises a clamping ring B which is coupled to the injection and aspiration tubes 8 and 9. The clamping ring B is mounted so as to slide along the injection and aspiration tubes 8 and 9, making it possible to lift the ring until the injection and aspiration tubes 8 and 9 are clamped under the patient's chin, such that the oropharyngeal cannula 1 is held securely in position on the patient.

As is shown in FIGS. 1 and 2, the second portion 44 of the tube 4 projects from a face of the collar 3 that is intended to be on the outside when the oropharyngeal cannula 1 is fitted on the patient. The second portion 44 of the tube 4 comprises a notch E formed in an upper wall S′ of the second portion 44 of the tube 4. The second portion 44 of the tube 4 and the notch E have dimensions sufficient to receive an infrared sensor capable of performing a capnographic measurement of the main flow of air delivered via the main conduit 5.

In a variant not shown, the oropharyngeal cannula 1 can also comprise additional outputs for injection of dioxygen nasally. In this variant, the oropharyngeal cannula 1 comprises an additional conduit formed in the collar 3. The additional conduit is connected to the first auxiliary conduit 6 and extends in the collar as far as two additional output orifices. The additional output orifices are provided on a radial upper part of the collar 3, so as to be opposite the patient's nostrils when the cannula 1 is fitted. The two additional output orifices can be coupled to two short additional tubes which are inserted into the patient's nostrils when the cannula 1 is fitted, in order to inject dioxygen into the patient's nostrils.

In order to improve the removal of the moisture that is present in the aspiration tube 9, the aspiration tube 9 can comprise a portion a few centimeters long, 5 cm for example, of Nafion tube. This portion is preferably situated 2 cm from the collar 3.

FIGS. 7 to 10 show schematic views of a nasopharyngeal cannula 10 according to an embodiment of the invention. The features similar to those of FIGS. 1 to 6 have the same reference letters or the same reference numbers multiplied by 10.

FIG. 7 shows a schematic plan view of the nasopharyngeal cannula 10 comprising a body 20 made of flexible plastic, for example medical PVC, and formed by a collar 30 through which a tube 40 passes. The tube 40 comprises two free ends 410 and 420 between which extend a first portion 430 and a second portion 440 which are arranged on either side of the collar 30.

As is illustrated in FIG. 8, which shows the nasopharyngeal cannula 10 from FIG. 7 in longitudinal section in the plane VIII-VIII′, the first portion 430 of the tube 40 has a curved shape so as to permit its insertion into the nostril of a patient and as far as his pharynx, so as to pass behind the base of the tongue and thereby keep the pharynx open.

The body 20 comprises a main conduit 50 extending between the two free ends 410 and 420 of the tube 40. The main conduit 50 thus forms a fluid passage between the outside and the airways of the patient, and more particularly his pharynx, when the nasopharyngeal cannula 10 is fitted on the patient.

The collar 30 is intended to rest on the outside of the base of the patient's nostril once the nasopharyngeal cannula 10 has been inserted into the patient's nostril.

As is shown in FIGS. 7 and 8, the nasopharyngeal cannula 10 in this embodiment comprises a single auxiliary conduit 60 formed within the thickness of the body 20 separating the main conduit 50 from the outside of the body 20. The auxiliary conduit 60 extends from the collar 30 into a proximal part 430p of the first portion 430 of the tube 40 in order to open into the main conduit 50 via an opening 610.

As is shown in FIG. 7, and also in FIG. 9 which shows the nasopharyngeal cannula 10 from FIG. 7 in a sectional view in the plane IX-IX′, the auxiliary conduit 60 comprises a T-shaped junction 620 which connects a first portion 630 of the auxiliary conduit 60, extending in the first portion 430 of the tube 40 parallel to the longitudinal axis VIII-VIII′ as far as the opening 610, and a second portion of the auxiliary conduit 60 formed by two segments 640 that extend in the collar 30.

Each segment 640 extends as far as a separate input orifice 650, thus forming an angle of about 10° with the plane defined by the surface of the collar 30, the two input orifices 650 being arranged on either side of the longitudinal axis VIII-VIII′ of the main conduit 50. The orientation of the two segments 640 of the second portion of the auxiliary conduit 60 allows the dioxygen injection tubes 80, connected to the input orifices 650, to be oriented toward the patient's face, thereby optimizing the maintenance in position of the nasopharyngeal cannula 10.

In order to optimize the injection of dioxygen into the main conduit 50, the auxiliary conduit 60 has a frustoconical shape at its end that opens into the opening 610. The cross section of the opening 610 is thus larger than the mean cross section of the auxiliary conduit 60. This frustoconical shape makes it possible to assist the good distribution of the fluid injected in the main conduit 50.

The upper wall S of the first portion 430 of the tube 40 has a rounded shape A at the free end 410. The rounded shape A makes it possible to reduce the risks of injury during the introduction of the nasopharyngeal cannula 10 into the patient's nostril.

The connection of the injection tubes 80 to the input orifices 650 is produced by welding or by overmolding the collar 30 onto the injection tubes 80, or with the aid of connectors.

The free end of each injection tube 8 can comprise a specific connector, for example a standard conical connector for dioxygen, which is designed to be coupled to a device delivering a flow of dioxygen, optionally via a tube cross section reducer.

As is shown in FIG. 9, each of the segments 640 of the second portion of the auxiliary conduit 60 forms an angle a with an axis passing through the T-shaped junction 620. The angle a has a value of between −10° and 20° and is preferably close to 0°. This angle allows the injection tubes 80 each to be oriented in the direction of the upper edge of an ear of the patient, so as to facilitate the passage of the injection tubes 80 behind the ears, as is illustrated in FIG. 10, which shows a schematic representation of the nasopharyngeal cannula 10 from FIG. 7 fitted on a patient. A negative value of the angle a or a value thereof close to zero allows the tubes to be passed under the nasal wings before being directed toward the ears. The passage of the injection tubes 80 behind the patient's ears allows the nasopharyngeal cannula 10 to be kept in place on the patient's nostril.

The nasopharyngeal cannula 10 additionally comprises a clamping ring B coupled to the injection tubes 80. The clamping ring B is mounted so as to slide along the injection tubes 80, so as to be able to lift the clamping ring B until the injection tubes 80 are clamped under the patient's chin, such that the nasopharyngeal cannula 10 is secured in position on the patient.

As is shown in FIGS. 7 and 8, the second portion 440 of the tube 40 projects from a face of the collar 30 that is intended to be on the outside when the nasopharyngeal cannula 10 is fitted on the patient. The second portion 440 of the tube 40 comprises a notch E formed in an upper wall S′ of the second portion 440 of the tube 40. The second portion 440 of the tube 40 and the notch E have dimensions sufficient to receive an infrared sensor capable of performing a capnographic measurement of the main flow of air delivered via the main conduit 50.

The embodiments presented above for an oropharyngeal cannula and for a nasopharyngeal cannula are not in any way limiting, and the features presented for an oropharyngeal cannula can be provided for a nasopharyngeal cannula, and vice versa.

The proposed invention provides an oropharyngeal or nasopharyngeal cannula which is designed to perform main-stream capnography. The proposed invention also makes it possible to provide a cannula that can be easily fixed and maintained on the patient in a removable manner.

Moreover, the proposed cannula is produced as a single piece or is preassembled, such that all that remains is to connect the injection/extraction tubes to the intended appliances. This makes it possible to reduce the fitting time and the risks of connection errors.

1. An oropharyngeal or nasopharyngeal cannula (1, 10) comprising a body (2, 20) formed by a tube (4, 40) extending through a collar (3, 30) that is able to rest on the outer edges of an oral or nasal cavity, respectively, of a patient, the tube (4, 40) comprising, on one side of the collar (3, 30), a first portion (43, 430) that is at least partially curved so as to be inserted into the oral or nasal cavity, respectively, of the patient as far as the pharynx, and a main conduit (5, 50) forming a fluid passage between the two ends (41 and 42; 410 and 420) of the tube (4, 40), characterized in that the tube (4, 40) comprises a rigid second portion (44, 440) arranged on the side of the collar (3, 30) opposite the first portion (43, 430), the second portion (44, 440) having a notch (E) which is transverse with respect to the axis (II-II′, VIII-VIII′) of the main conduit (5, 50) and which is configured to receive infrared means that are able to perform a capnographic measurement of the air exhaled through the main conduit (5, 50).

2. The cannula (1, 100) as claimed in claim 1, in which the collar (3, 30) comprises two input/output orifices (65 and 75, 650) which are oriented in a radial direction with respect to the axis (II-II′, VIII-VIII′) of the main conduit (5, 50) in such a way as to be oriented laterally on each side of the oral or nasal cavity of the patient when the cannula (1, 10) is fitted on the patient, the two input/output orifices (65 and 75, 650) being connected to one or two auxiliary conduits (6 and 7, 60) which are formed in the body (2, 20) and extend from the collar (3, 30) to the main conduit (5, 50) via the first portion (43, 430) of the tube (4, 40).

3. The cannula (1, 10) as claimed in claim 2, in which the two input/output orifices (65 and 75, 650) arranged radially on the collar (3, 30) are connected to an auxiliary conduit (6, 7, 60) via a portion (64, 74, 640) of the auxiliary conduit (6, 7, 60) extending radially in the collar (3, 30), each forming an angle of between 0° and 20°, more particularly an angle of about 10°, with the plane defined by the surface of the collar (3, 30), so as to orient the input/output orifices (65 and 75, 650) toward the patient's face.

4. The cannula (1, 10) as claimed in claim 2 or 3, comprising two flexible injection/extraction tubes (8 and 9, 80) which are able to be passed behind the patient's ears when the cannula (1, 10) is fitted on the patient, a first end of each tube being connected to an input/output orifice (65 and 75, 650).

5. The cannula (1, 10) as claimed in claim 4, comprising a clamping ring (B) which is able to hold the two injection/extraction tubes (8 and 9, 80) together and can slide along the two injection/extraction tubes (8 and 9, 80) so as to hold the cannula (1, 10) in place when it is fitted on the patient and each injection/extraction tube (8 and 9, 80) passes behind an ear.

6. The cannula (1, 10) as claimed in either of claims 4 and 5, in which a second end of the injection/extraction tubes (8 and 9, 80) comprises means for connection to injection or extraction appliances.

7. The cannula (1, 10) as claimed in one of claims 4 through 6, comprising a single auxiliary conduit (6, 60) which extends as far as a proximal part (43p, 430p) of the first portion (43, 430) of the tube (4, 40) and is connected via the two input/output orifices (65 and 75, 650) to the injection/extraction tubes (8 and 9, 80), said injection/extraction tubes (8 and 9, 80) being tubes for injection of dioxygen.

8. The cannula (1, 10) as claimed in one of claims 4 through 6, comprising a first auxiliary conduit (6) which extends as far as a proximal part (43p) of the first portion (43) of the tube (4), and a second conduit (7) extending as far as a distal part (43d) of the first portion (43) of the tube (4), the first auxiliary conduit (6) being connected via an input orifice (65) to an injection tube (8) for dioxygen, and the second auxiliary conduit (7) being connected to an output orifice (75), which is connected to a tube (9) for aspiration of the secretions in the patient's airways.

9. The cannula (1, 10) as claimed in claim 8, in which the second auxiliary conduit (7) is formed in an upper wall (S) of the first portion (43) of the tube (4).

10. The cannula (1, 10) as claimed in one of claims 2 through 9, in which at least one of the auxiliary conduits (6, 7, 60) comprises a cross section of oblong shape, at least on part of the first portion (43, 430) of the tube (4, 40).

11. The cannula (1, 10) as claimed in one of claims 2 through 10, in which at least one of the auxiliary conduits (6, 7, 60) comprises an orifice which opens into the main conduit (5, 50) and has a frustoconical shape, the orifice having a larger cross section than the mean cross section of the auxiliary conduit (6, 7, 60).

12. The nasopharyngeal cannula (10) as claimed in one of claims 2 through 11, in which the direction defined by an input/output orifice (650) and the axis (II-II′) of the main conduit (50) forms an angle of between −10° and 20°, more particularly between 0° and 10°, with an axis orthogonal to the axis of the patient's nose when the cannula (10) is fitted on the patient.

13. The nasopharyngeal cannula (10) as claimed in one of claims 2 through 12, in which the free end (410) of the first portion (430) of the tube (40) is provided with a rounded shape at least on its upper wall (S).

14. The nasopharyngeal cannula (10) as claimed in one of claims 2 through 13, in which the body (20) is made of flexible and sliding plastic.

15. The oropharyngeal cannula (1) as claimed in one of claims 2 through 11, in which the direction defined by an input/output orifice (65 and 75) and the axis (II-II′) of the main conduit (50) forms an angle of between 0° and 30°, more particularly between 10° and 20°, with an axis passing through the corners of the patient's lips when the cannula (1) is fitted on the patient.

16. The oropharyngeal cannula (1) as claimed in one of claims 2 through 11 or 15, comprising an additional conduit formed in the collar (3) and extending from a portion (64) of an auxiliary conduit (6) as far as two additional output orifices that are formed in an upper part of the collar (3), the two additional output orifices being arranged so as to be opposite each of the patient's nostrils when the cannula (1) is fitted on the patient.
